(19) United States
(12) Patent Application Publication
(10) Pub. No.: US 2026/0272839 A1
(43) Pub. Date: Sep. 17, 2026

(54) COMPOSITION AND METHOD FOR IMPROVING DIETARY SUPPLEMENT SOLUBILITY AND BIOAVAILABILITY VIA LIPOSOMAL DELIVERY

(71) Applicant: Spartacus Brands LLC, Boca Raton, FL (US)

(72) Inventors: Augustin Quancard, Boca Raton, FL (US); Gregory Papigny, Boca Raton, FL (US); Jeremy Eisenberg, Boca Raton, FL (US); Stephanie Eisenberg, Boca Raton, FL (US); Gary Bitton, Boca Raton, FL (US)

(21) Appl. No.: 19/654,253

(22) Filed: Apr. 21, 2026

Related U.S. Application Data
(63) Continuation-in-part of application No. 19/081,561, filed on Mar. 17, 2025.

(51) Int. Cl. A61K 9/1277

(52) U.S. Cl. CPC A61K 9/1277

(57) ABSTRACT

A powdered composition and method are provided to enhance the delivery and bioavailability of supplemental nutrients, metabolites, bioactive compounds, therapeutic agents, and/or combinations of the same as dietary supplements through liposomal encapsulation. The method comprises mixing such supplemental nutrients with inactive ingredients such as anti-caking agents, preservatives, stabilizers, and/or emulsifiers and, to the mixture, adding and further blending a phospholipid material such as sunflower lecithin to liposomally encapsulate the supplemental nutrient particles. The resulting composition improves texture, shelf life, and even dispersion in liquid. By encapsulating dietary supplements, the liposomal blend protects the dietary supplements from degradation in the digestive system, enhancing absorption and facilitating the passage of encapsulated dietary supplements across the intestinal wall and into the bloodstream, improving systemic delivery to target regions in the body.

### CROSS-REFERENCE TO RELATED APPLICATIONS

This application is a continuation-in-part of U.S. patent application Ser. No. 19/081,561 filed on Mar. 17, 2025, the entire contents of which are hereby incorporated by reference.

### GOVERNMENT CONTRACT

Not applicable.

### STATEMENT RE. FEDERALLY SPONSORED RESEARCH/DEVELOPMENT

Not applicable.

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the Patent and Trademark Office patent files or records, but otherwise reserves all copyrights and trade dress rights whatsoever.

### TECHNICAL FIELD

The disclosed subject matter relates generally to nutrient delivery and more particularly to targeted nutrient delivery of ingestible dietary supplements using liposomes.

### BACKGROUND

Improving the solubility and bioavailability of supplements is highly desirable in the development of novel formulations, particularly for compounds with poor water solubility that exhibit limited absorption in the body. Many bioactive ingredients, such as vitamins, polyphenols, and plant-derived compounds, face significant challenges in delivering their intended benefits due to their low solubility, poor permeability, and rapid degradation in the gastrointestinal tract.

Enhancing solubility through advanced delivery technologies, such as liposomal encapsulation, nanoemulsions, or micellar systems, allows for greater dissolution in biological fluids, leading to improved absorption and increased systemic availability. This, in turn, enables lower dosages to achieve the desired therapeutic effect, reduces variability in individual responses, and minimizes potential side effects associated with high-dose consumption.

Some proposals to further improve the solubility and bioavailability of dietary supplements have been made. For instance, U.S. Pat. Pub. No. 2007/0178147 to Desai et al. teach liposomal pharmaceutical compositions, and U.S. Pat. No. 11,684,575 to Ono et al. teaches a liposome composition having a practically required long term preservation stability for delivering anti-cancer drugs. Indeed, the inventor's own disclosures, which are herein incorporated by reference, relate to liposomal delivery of certain dietary supplements, including, for instance, creatine in U.S. Pat. Pub. No. 2024/0156732 by Quancard et al. and urolithin in U.S. Pat. Pub. No. 2024/0261251 by Eisenberg et al.

However, these proposals remain deficient in effectively enhancing the solubility and bioavailability of a broader range of dietary supplements. In addition, many existing liposomal formulations are optimized for pharmaceutical applications rather than dietary supplements, often requiring complex manufacturing processes and formulation strategies that do not fully address the challenges specific to nutraceuticals. Additionally, issues such as long-term stability and formulation constraints can limit their broader applicability for general consumption.

Thus, although various proposals have been made to address the bioavailability of supplements by liposomal delivery, there remains a need for improved formulations that can overcome these limitations while maintaining high bioavailability, stability, and practicality for consumer use in powdered, water dispersible form.

### SUMMARY AND OBJECTS OF THE INVENTION

The present disclosure is directed to a composition for increased absorption of dietary supplements via liposomal delivery. More particularly, the composition is a powder configured for suspension in a liquid and comprises a liposomal blend of phospholipids sized and activated to effectively improve dietary supplement solubility and bioavailability in a body by preventing premature degradation within the digestive system and facilitating the direct release of the supplement into a targeted region.

For purposes of summarizing, certain aspects, advantages, and novel features have been described. It is to be understood that not all such advantages may be achieved in accordance with any one particular embodiment. Thus, the disclosed subject matter may be embodied or carried out in a manner that achieves or optimizes one advantage or group of advantages without achieving all advantages as may be taught or suggested.

In accordance with one embodiment, the liposomal blend configured for liposomal delivery of supplemental nutrients, metabolites, and/or other bioactive compounds and therapeutic agents (or “dietary supplement(s)”) may comprise liposomes, including, for example, and without limitation, phospholipids, eggs, or any other form of liposomes. One of ordinary skill in the art will recognize that liposomes are a type of artificial vesicle comprising a hydrophobic membrane of at least one lipid bilayer and an aqueous core. Phospholipids, liposomes, and lipids bilayers are all known in the art, and in the interest of brevity, only a brief description of such components has been provided.

Phospholipids comprising the liposomal blend may be any phospholipids known in the art, for example, any of phosphatidylcholine, phosphorylethanolamine, phospholipid, or phosphatidylserine. In one embodiment, the phospholipids may be plant-based. For example, in an embodiment wherein the phospholipids is phosphatidylcholine, the phosphatidylcholine may be extracted from sunflowers. In some embodiments, the phospholipids are sunflower lecithin.

It is contemplated that by using plant-based phospholipids, the composition may be vegetarian, or even vegan, and thus may be available to those with specific dietary needs. Of course, other forms of phospholipids may be utilized.

A person of ordinary skill in the art will recognize the manners in which liposomes may bind to a subject's cell membrane(s). It is contemplated that the liposomal blend being operative to bind to the cell membrane may permit dietary supplement(s), such as supplemental nutrients, metabolites, and/or other bioactive compounds and therapeutic agents, encapsulated within the liposomal blend to pass directly into a targeted region of the user's body, as such, increasing absorption of any encapsulated dietary supplements. Thus, the liposomal blend may protect the supplemental nutrients, metabolites, and/or other bioactive compounds and therapeutic agents from degradation that occurs due to the digestive system.

A method for preparing a liposomal supplement formulation configured to improve solubility, dispersibility, and delivery of dietary supplements is also provided. Some embodiments of this method may comprise introducing one or more active ingredients into a mixing vessel, adding one or more inactive ingredients (such as the dietary supplements) and/or fillers (such as anti-caking agents, preservatives, stabilizers, and emulsifiers), and mixing the ingredients to achieve a substantially uniform mixture. A phospholipid component, such as a water-dispersible phospholipid material such as sunflower lecithin, may then be added to the mixture and blended for a period sufficient to distribute the phospholipid component and promote formation of liposomal particles. In certain embodiments, the mixing may be performed using a ribbon blender, such as a double-helix ribbon blender equipped with variable speed control to promote uniform dispersion of the ingredients while minimizing particle agglomeration.

It is contemplated that formulations prepared according to the disclosed method may produce liposomal particles having particle sizes within a liposomal particle size range suitable for stable aqueous dispersion. In some embodiments, the resulting formulation may be evaluated using particle size analysis or dispersion testing to verify that the particles fall within a liposomal particle size range and remain stably dispersed in aqueous media.

In some embodiments, the phospholipids are provided as a water dispersible powder. That is, the composition is configured to form an even suspension in water, in order to maintain protection of any dietary supplements contained therein, as described above. To that end, the phospholipids may be combined with one or more anti caking agents, preservatives, stabilizers, and/or emulsifiers operative to improve the texture and shelf-life of the phospholipid powder as well as the texture of any liquid mixed with the phospholipid powder for oral consumption.

The dietary supplement may be an essential nutrient such as essential vitamins and/or minerals that the body cannot synthesize in sufficient quantities on its own. Thus, in some embodiments, the dietary supplement may be one or more vitamins, such as vitamins A, C, D, E, K and B-complex vitamins, among others, known to those of ordinary skill in the art. In some exemplary embodiments, the dietary supplement may be a fat-soluble nutrient such as vitamins A, D, E, and K, which require fat for improved uptake. Still, water soluble vitamins such as vitamin C and B-complex vitamins, like vitamin B12, folate, and niacin may also benefit from liposomal delivery by increasing their bioavailability and reducing gastrointestinal irritation.

Likewise, some essential minerals such as iron, magnesium, zinc, and selenium can be delivered with liposomes to improve their solubility and reduce gastrointestinal side effects. Indeed, providing delivering these minerals with liposomal compositions can protect them from degradation and enhance their absorption in the intestines.

Similarly, it is contemplated that delivering essential fatty acids, such as omega-3s like DHA and EPA, via the liposomal composition can improve their stability, prevent oxidation, and enhance cellular update. This is particularly true of these fatty acids that are known to have low bioavailability and be prone to degradation. Of course, other supplemental nutrients, metabolites and other bioactive compounds, therapeutic agents, and combinations of the same, can be delivered according to this disclosure as needed or desired without departing from the invention. Overall, it is contemplated that providing liposomal delivery of nutrients, metabolites, and other bioactive compounds and therapeutic agents, including combinations of the same according to the invention will provides a significant advantage for such supplements by protecting them from degradation, enhancing their solubility and bioavailability, and improving their stability during storage and digestion.

In some embodiments, it is contemplated that the liposomal blend may be operative to control the release of the encapsulated dietary supplement. Controlling the release of such supplement may alter the period of time in which the supplement is effective within a subject's body. Further, controlled release may increase the half-life of such supplement, extending an effective period of the supplement within the body.

It is still further contemplated that the liposomal blend may comprise ingredients such as one or more anti caking agents, preservatives, stabilizers, and emulsifiers in effective amounts to improve the texture and shelf-life of the liposomal blend and indeed the powder composition.

It is contemplated that formulating a composition for increased absorption of supplemental nutrients according to the disclosure and claims provided below may have the following advantages:
a.) increased absorption of supplemental nutrients, metabolites and other bioactive compounds and therapeutic agents;
b.) improves the bioavailability of supplemental nutrients, metabolites and other bioactive compounds and therapeutic agents;
c.) reduces waste of ingested supplemental nutrients, metabolites and other bioactive compounds and therapeutic agents caused by the digestive system;
d.) controls the release of supplemental nutrients, metabolites and other bioactive compounds and therapeutic agents; and
e.) increases the stability of supplemental nutrients, metabolites and other bioactive compounds and therapeutic agents.

It is further contemplated that preparing embodiments of the composition according to the methods disclosed herein may have the following advantages:
a.) producing a formulation in which substantially all particles fall within a liposomal particle size range, thereby producing a 100% liposomal formulation when measured using particle size analysis;
b.) increased absorption of supplemental nutrients, metabolites and other bioactive compounds and therapeutic agents;
c.) improved bioavailability of supplemental nutrients, metabolites and other bioactive compounds and therapeutic agents;
d.) reduced waste of ingested supplemental nutrients, metabolites and other bioactive compounds and therapeutic agents caused by the digestive system;
e.) controlled release of supplemental nutrients, metabolites and other bioactive compounds and therapeutic agents; and
f.) increased stability of supplemental nutrients, metabolites and other bioactive compounds and therapeutic agents.

Thus, it is an object of this invention to reduce dietary supplement waste.

It is yet another object of this invention to increase the shelf-life of dietary supplements.

It is still another object of this invention to improve even suspension of dietary supplements in powdered form in liquids for oral consumption.

It is yet a further object of this invention to distribute dietary supplements to targeted areas of the body.

One or more of the above-disclosed embodiments, in addition to certain alternatives, are provided in further detail below. The disclosed subject matter is not, however, limited to any particular embodiment disclosed.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “couple,” “coupled,” “couples,” “coupling,” and the like should be broadly understood and refer to connecting two or more elements or signals, electrically, mechanically or otherwise. Two or more electrical elements may be electrically coupled, but not mechanically or otherwise coupled; two or more mechanical elements may be mechanically coupled, but not electrically or otherwise coupled; two or more electrical elements may be mechanically coupled, but not electrically or otherwise coupled. Coupling (whether mechanical, electrical, or otherwise) may be for any length of time, e.g., permanent or semi-permanent or only for an instant.

### BRIEF DESCRIPTION OF THE DRAWINGS

FIG. 1 is a flowchart depicting one embodiment of a method for improving dietary supplement solubility and bioavailability via liposomal delivery.

The disclosed embodiments may be better understood by referring to the FIGURES in the attached drawings, as provided below. The attached FIGURES are provided as non-limiting examples for providing an enabling description of the method and system claimed. Attention is called to the fact, however, that the appended drawings illustrate only typical embodiments of this invention and are therefore not to be considered as limiting of its scope. One skilled in the art will understand that the invention may be practiced without some of the details included in order to provide a thorough enabling description of such embodiments. Well-known structures and functions have not been shown or described in detail to avoid unnecessarily obscuring the description of the embodiments.

### DETAILED DESCRIPTION

Having summarized various aspects of the present disclosure, reference will now be made in detail to various embodiments of the composition. While the disclosure will be described in connection with these embodiments, there is no intent to limit it to the embodiment or embodiments disclosed herein. Rather, the intent is to cover all alternatives, modifications and equivalents included within the spirit and scope of the disclosure as defined by the appended claims.

In accordance with one embodiment, the composition may comprise one or more supplemental nutrients, metabolites, bioactive compounds, therapeutic agents, and/or combinations of the same as dietary supplements encapsulated within a liposomal blend comprising, in part, liposomes, which are a chemical structure that will be understood by a person of ordinary skill in the art. In the interest of clarity, non-limiting examples and embodiments of liposomes are discussed, though any form or embodiment of liposomes may be utilized.

The liposomes in the liposomal blend may be any liposomes known in the art. In general, liposomes comprise at least on lipid bilayer surrounding an aqueous core. The lipid bilayer may be formed from phospholipids, which comprise a hydrophilic head and a hydrophobic tail. Liposomes are generally spherical in shape and will be discussed as such for the sake of brevity, however, liposomes may vary in shape and form, and any shape or form may be utilized. A person of ordinary skill in the art will appreciate that liposomes may contain an active ingredient, such as the dietary supplement or supplements, in the aqueous core or between the hydrophobic tails of the lipid bilayer. While the dietary supplement(s) may be contained in either, or even both, of the aforementioned locations, in the interest of brevity, reference, is made to the embodiments wherein the dietary supplement(s) is contained in the aqueous core.

A person of ordinary skill in the art will appreciate that encapsulating the dietary supplement(s) in the liposomes may be done in any number of manners, and the subsequent embodiments are provided as non-limiting examples only. Generally, preparing the liposomes will comprise drying lipids, dispersing the lipids in an aqueous media to form the liposomes, and purifying the liposome. The aqueous media may comprise the dietary supplement(s) that, when encapsulated, may form the aqueous core.

In one embodiment, the dietary supplement(s) may be encapsulated in the liposomes through a passive loading method. For example, the passive loading method may mechanically disperse the dietary supplement(s) through any of a mechanical dispersion method, a solvent dispersion method, and a detergent removal method. Of course, other forms of passive loading may be utilized, and the aforementioned are provided as examples only. In the interest of clarity, one embodiment of mechanical dispersion is provided, though any form of passive encapsulation may be utilized. Mechanical dispersion may utilize a process known in the art as sonication, where the lipids and aqueous media are exposed to high-frequency sound waves. It is contemplated that sonication may be operative to create a more homogenous suspension and may reduce large particles.

In another embodiment, the dietary supplement(s) may be encapsulated in the liposomes through active loading. Active loading may occur following the formation of liposomes and may permit the dietary supplement(s) to be added at a later time. In some embodiments, active loading may be accomplished using gradient loading, which involves a buffer that uses a PH gradient for loading the dietary supplement(s) into the liposome. For example, the aqueous core of the liposome may comprise a low PH and may be suspended in a solution comprising a high PH and the dietary supplement(s). The solution may permeate the liposome to equalize the PH, thus delivering the dietary supplement(s) into the aqueous core of the liposome.

In one embodiment, the phospholipids may be lecithin phospholipids. In some such embodiment, the lecithin phospholipids may be extracted from sunflowers. More particularly, the lecithin phospholipids may be extracted from sunflower oil. Of course, phospholipids may be extracted from a large number of plants and animals, such as from eggs, meat, seafood, and soy. As such, any form of phospholipids may be utilized, and the aforementioned are provided as non-limiting examples.

The terms “lecithin,” “phospholipids,” and “phosphatidylcholine” are used interchangeably. A person of ordinary skill in the art will appreciate that lecithin may comprise additional substances, such as triglycerides, fatty acids, and carbohydrates. While phospholipids and phosphatidylcholine may be generally understood to comprise a lower concentration of the additional substances, for the purposes of this disclosure, unless otherwise provided, the use of phospholipids and phosphatidylcholine is synonymous with lecithin.

In one embodiment, the composition may comprise between about 90 to about 95% by weight of a dietary supplement or combination of dietary supplements in addition to no more than about 5% by weight phospholipids as the liposomes. This is because liposomal particle formation and stability may be influenced by the concentration of phospholipids within the composition. For instance, it should be noted that in a powdered composition, more than about 5% by weight phospholipids may result in undesirable foaming in a liquid suspension and decrease efficacy of the liposomal blend. In some embodiments, phospholipids may be present in an amount of approximately 1.5% to approximately 3% by weight of the composition, which has been observed to provide stable liposomal particle formation while minimizing foaming in suspension. In addition, the phospholipid particle size may be up to about 1,000 nanometers as determined by dynamic light scattering analysis showing consistent particle distribution and confirming that phospholipid particles remain stable in a liquid solution overtime. It will be understood that 1,000 nm corresponds to a liposomal particle size range that supports stable dispersion of the formulation in aqueous media.

As used herein, the phrase “100% liposomal” refers to a formulation in which substantially all particles measured using dynamic light scattering (DLS) fall within a liposomal particle size range, for example less than or equal to approximately 1,000 nanometers, indicating that the formulation predominantly comprises liposomal particles rather than non-liposomal aggregates.

In addition to the phospholipids, the liposomal blend may comprise one or more anti caking agents, preservatives, stabilizers, and emulsifiers in effective amounts to improve the texture and shelf-life of the phospholipid powder composition. These may comprise, for example only and not limitation, silicon dioxide, polysorbate 80, polypropylene glycol, and acetylated monoglycerides, all known to those of ordinary skill in the art.

As such, in some embodiments, the liposomal blend may comprise about 45 to about 60% by weight phospholipids, about 25 to about 40% by weight silicon dioxide, about 10 to about 25% by weight polysorbate 80, up to about 10% by weight propylene glycol, and up to about 10% by weight acetylated monoglycerides.

Thus, in accordance with some embodiments, a powdered composition may comprise between about 90 to about 95% by weight of the dietary supplement, 1.5 to about 3% by weight phospholipid, about 1.0 to about 2.0% by weight silicon dioxide, about 0.1 to about 1.0% by weight polysorbate 80, about 0.1 to about 0.5% by weight polypropylene glycol, and about 0.1 to about 0.5% by weight acetylated monoglycerides.

In some embodiments, the composition may comprise additional ingredients, including, for example, and without limitation, flavoring, coloring, or even other dietary supplements as described herein.

In some embodiments, the composition may be ingestible. For example, the composition may be orally consumed. It is contemplated that the composition may be dispersible in water. Of course, other liquids may be utilized, such as juice, coffee, milk, or even other liquids or combinations thereof. It is contemplated that the composition may be at least partially dissolvable and even be dissolved in the same liquids as water-soluble solutes, for example, electrolytes, vitamins, or flavoring.

In another embodiment, the composition may be topically applied. In yet another embodiment, the composition may be intravenously delivered. Of course, the composition may be in any form capable of being delivered into the body.

In some embodiments, the composition may comprise flavoring. The flavoring may be artificial or natural flavoring and be present at any concentration as needed or desired. Of course, in other embodiments, the composition may be unflavored. In yet another embodiment, the composition may comprise natural or artificial coloring.

In yet another embodiment, the composition may comprise additional dietary supplements, such as nutrients, metabolites, bioactive compounds, therapeutic agents, or combination of the same. For example, the composition may comprise supplemental proteins, amino acids, vitamins, antioxidants, minerals, fibers, or any other dietary supplements that may be needed or desired. It is contemplated that the additional dietary supplements may be present at any concentration in the composition as needed or desired.

In some embodiments, the compositions described herein may be prepared according to a controlled method configured to produce stable liposomal particles having particle sizes within the ranges described above. Embodiments of the method may include controlled mixing of active ingredients, inactive ingredients, and phospholipid materials using equipment capable of reliably producing uniform dispersion of the ingredients and minimizing particle agglomeration.

Experimental testing conducted during development of the methods described herein demonstrated that the characteristics of the phospholipid component and the specific manufacturing process parameters significantly influence the consistency with which liposomal particles are produced. While liposomal particles may be formed using various phospholipid materials and preparation methods, it was observed that many alternative phospholipid materials, equipment configurations, and manufacturing sequences produced liposomal particles only intermittently or with inconsistent particle size distributions. In contrast, formulations prepared according to the methods described herein consistently produced liposomal particles within the desired particle size range as measured by dynamic light scattering analysis (described in further detail below).

In particular, the use of water-dispersible sunflower-derived lecithin having the compositional characteristics described herein, combined with the disclosed concentration ranges and manufacturing sequence, was observed to produce stable liposomal particles with greater reproducibility and stability in aqueous suspension. These results indicate that the phospholipid characteristics and manufacturing parameters disclosed herein cooperate to improve the reliability and stability of liposomal particle formation.

In some embodiments, the compositions may be prepared using a ribbon blender comprising a double helix mixing element. The ribbon blender may be equipped with variable speed drives allowing the mixing speed to be adjusted depending on the formulation characteristics. Such equipment has been observed to promote uniform distribution of ingredients while reducing the likelihood of particle agglomeration during mixing.

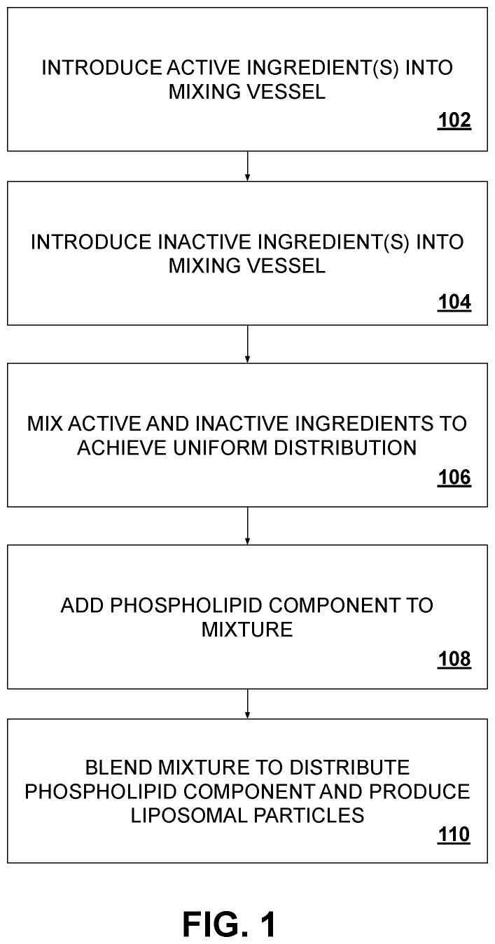

In some embodiments, raw materials may be added to the mixing equipment in a controlled sequence. Turning to FIG. 1, active ingredients may be introduced first into the mixing vessel (block 102). Inactive ingredients or fillers, such as anti-caking agents, preservatives, stabilizers, and emulsifiers, may then be added (block 104). These components may be mixed (block 106) for a period sufficient to achieve uniform distribution, which in some embodiments may be approximately 10-15 minutes depending on the formulation. In some embodiments, this may result in a homogenous mixture.

Following initial mixing, the phospholipid material or component may be added to the mixture (block 108). In some embodiments, a phospholipid component comprising, for example, water-dispersible sunflower lecithin having the compositional characteristics described above may be used. The phospholipid component may be added as the final ingredient in order to minimize the risk of particle agglomeration during mixing.

After addition of the phospholipid component, the mixture may be further blended (block 110) for an additional mixing period, which in some embodiments may be approximately 20 minutes, although mixing times may vary depending on formulation characteristics and batch size. The objective of the mixing process is to achieve uniform distribution of ingredients while avoiding excessive mixing that may promote particle agglomeration.

In some embodiments, quality control testing may be conducted during or after mixing to confirm homogeneity of the mixture and to evaluate liposomal particle formation. Such testing may include particle size measurements, dispersibility testing, or dynamic light scattering analysis to verify that the resulting formulation produces particles within the desired liposomal particle size range.

It will be understood that adjustments to the method, such as mixing duration(s), may be made depending on the characteristics of the active ingredients and excipients used in the formulation without departing from the invention.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

### EXAMPLES OF THE PREFERRED EMBODIMENT OF THE COMPOSITION

In order to more fully teach what the Applicant regards as their invention, the following example is given. It should be understood that the formulation set forth in the Example is not to be construed as limiting the scope of the invention, except so far as they yield a composition having the desired properties and characteristics. More particularly, and by way of example, the following chart illustrates a formulation of the same with percentages given by weight of the composition.

### Example 1

### Liposomal Blend:

| Ingredient | Percentage |
|---|---|
| Sunflower lecithin | 50.0 |
| Silicon Dioxide | 30.0 |
| Polysorbate 80 | 10.0 |
| Polypropylene Glycol | 5.0 |
| Acetylated Monoglycerides | 5.0 |
| TOTAL: | 100.0% |

### Example 2

### Dietary Supplement:

| Ingredient | Percentage |
|---|---|
| Vitamin C | 94.0 |
| Sunflower Lecithin | 2.9 |
| Silicon Dioxide | 1.9 |
| Polysorbate 80 | 0.6 |
| Polypropylene Glycol | 0.3 |
| Acetylated Monoglycerides | 0.3 |
| TOTAL: | 100.0% |

### EXPERIMENTAL RESULTS

In order to determine the efficacy of the methods described herein for producing liposomal supplement formulations, experimental testing was performed on multiple formulations prepared according to the disclosed manufacturing process. The testing was conducted to validate that the disclosed phospholipid composition, ingredient concentration ranges, and manufacturing procedures consistently produce liposomal particles meeting predetermined particle size and stability criteria while also improving dispersibility and solubility relative to non-liposomal formulations.

The disclosed manufacturing method was validated using two principal analytical tests performed repeatedly across a variety of supplement formulations prepared according to the methods described herein. These tests comprised: (i) a liposomal formation test evaluating particle size distribution and phospholipid stabilization characteristics, and (ii) a dispersibility and solubility test evaluating the ability of the resulting formulation to disperse and dissolve in aqueous media. The results of these tests demonstrated that formulations prepared according to the disclosed method consistently produced liposomal particles within defined size ranges and exhibited improved solubility relative to comparable non-liposomal formulations.

### Experiment 1—Liposomal Formation Test

A liposomal formation test was conducted to evaluate whether formulations prepared according to the disclosed method produced particles meeting established liposomal size and stability criteria. Liposomal particles were evaluated based on particle size distribution and phospholipid content within the formulation. Particle size and stability were measured using dynamic light scattering (DLS), a technique known to those of ordinary skill in the art as capable of measuring particle dispersion and stability in liquid suspensions.

In this test, liposomal particles were characterized according to particle size ranges measured by DLS analysis. In some embodiments, particles having a diameter of less than or equal to approximately 1,000 nanometers were considered suitable liposomal particles. Particles having diameters between approximately 1,000 and 5,000 nanometers were considered oversized and may indicate incomplete liposomal formation, while particles greater than approximately 5,000 nanometers were considered non-liposomal aggregates.

Stability of the particle suspension was further evaluated by measuring the degree of particle dispersion and resistance to agglomeration. DLS analysis enabled monitoring of particle stability in aqueous solution by determining whether particles remained dispersed or agglomerated over time. In these evaluations, it was observed that the selection and characteristics of the phospholipid component used in the formulation were critical for maintaining particle stability in solution. In particular, phospholipids having water-dispersible characteristics, such as sunflower-derived lecithin, were observed to provide effective surface activation of the particles, allowing the particles to remain dispersed in aqueous media and maintain what may be described as aqueous singularity, thereby preventing particle agglomeration.

While the selection and properties of the phospholipid component were found to be particularly important for achieving stable liposomal formation, the concentration of phospholipids within the formulation also influenced particle stability and performance. In some embodiments, phospholipids may be present in an amount between approximately 1.5% and approximately 3% by weight of the composition. Concentrations within this range were observed to provide sufficient surface activation of the particles while maintaining stable liposomal dispersions. In some instances, phospholipid concentrations greater than approximately 5% by weight were observed to produce excessive foaming when suspended in liquid, which may negatively affect formulation performance.

The liposomal formation test was conducted on multiple formulations produced using the method described herein. The results consistently demonstrated that formulations prepared according to the disclosed method produced liposomal particles within the desired size range of less than or equal to approximately 1,000 nanometers. In contrast, variations of the method, including the use of alternative phospholipid blends, alternative equipment, or phospholipid concentrations outside the disclosed ranges, frequently resulted in the formation of particles outside the desired liposomal size range or resulted in particle agglomeration.

The results therefore confirmed that the disclosed combination of phospholipid composition, concentration ranges, and manufacturing process parameters consistently produces liposomal formulations exhibiting stable particle dispersion and particle sizes within the liposomal range.

### Experiment 2—Dispersibility and Solubility Test

A dispersibility and solubility test was also conducted to evaluate the ability of formulations prepared according to the disclosed liposomal manufacturing method to disperse in aqueous solutions and improve solubility of the active compounds contained therein. The dispersibility characteristics of the formulation were evaluated using DLS analysis to assess particle distribution and polydispersity in aqueous media.

Polydispersity values obtained through DLS analysis were used to evaluate whether the liposomal particles remained effectively dispersed in solution. Proper dispersibility of particles in aqueous media is considered an important factor in improving the bioavailability of compounds delivered through liposomal systems, as dispersion allows greater surface interaction with biological membranes and improves absorption potential.

Comparative testing was performed between liposomal formulations prepared according to the disclosed method and corresponding non-liposomal formulations containing the same active compounds. The results demonstrated that liposomal formulations produced according to the disclosed method exhibited increased solubility relative to the non-liposomal formulations. In certain tests, the liposomal formulations exhibited about 60% greater solubility compared to corresponding non-liposomal formulations containing the same active compounds. DLS measurements further indicated improved particle dispersion and reduced particle agglomeration in the liposomal formulations.

As such, the increased solubility and dispersibility of the liposomal formulations may facilitate improved absorption of the active compounds following ingestion. In some embodiments, the liposomal particles may provide a gradual release of the encapsulated compounds upon reaching the intestinal environment, thereby allowing additional time for absorption at the intestinal wall. Indeed, in certain experimental evaluations, liposomal formulations prepared according to the methods described herein demonstrated increased uptake in cellular models relative to comparable non-liposomal formulations, supporting the enhanced delivery characteristics of the liposomal system.

The dispersibility and solubility test results therefore demonstrate that formulations prepared according to the disclosed method provide improved dispersibility in aqueous environments and enhanced solubility relative to comparable non-liposomal formulations, supporting improved delivery and potential absorption of the active compounds contained therein.

### CONCLUSIONS, RAMIFICATIONS, AND SCOPE

While certain embodiments of the invention have been illustrated and described, various modifications are contemplated and can be made without departing from the spirit and scope of the invention. For example, the composition may be suitable to dissolve in any drink as needed or desired according to the taste and dietary preferences of a user. Accordingly, it is intended that the invention not be limited, except as by the appended claim(s).

The teachings disclosed herein may be applied to other systems and methods, and may not necessarily be limited to any described herein. The elements and acts of the various embodiments described above can be combined to provide further embodiments. All of the above patents and applications and other references, including any that may be listed in accompanying filing papers, are incorporated herein by reference. Aspects of the invention can be modified, if necessary, to employ the systems, functions and concepts of the various references described above to provide yet further embodiments of the invention.

Particular terminology used when describing certain features or aspects of the invention should not be taken to imply that the terminology is being refined herein to be restricted to any specific characteristics, features, or aspects of the composition for improving dietary supplement solubility and bioavailability via liposomal delivery with which that terminology is associated. In general, the terms used in the following claims should not be constructed to limit the composition for improving dietary supplement solubility and bioavailability via liposomal delivery to the specific embodiments disclosed in the specification unless the above description section explicitly define such terms. Accordingly, the actual scope encompasses not only the disclosed embodiments, but also all equivalent ways of practicing or implementing the disclosed system, method and apparatus. The above description of embodiments of the composition for improving dietary supplement solubility and bioavailability via liposomal delivery is not intended to be exhaustive or limited to the precise form disclosed above or to a particular field of usage.

While specific embodiments of, and examples for, the composition are described above for illustrative purposes, various equivalent modifications are possible for which those skilled in the relevant art will recognize.

While certain aspects of the method and system disclosed are presented below in particular claim forms, various aspects of the method, system, and apparatus are contemplated in any number of claim forms. Thus, the inventor reserves the right to add additional claims after filing the application to pursue such additional claim forms for other aspects of the composition for improving dietary supplement solubility and bioavailability via liposomal delivery.

What is claimed is:

1. A method of preparing a dietary supplement composition, comprising:
introducing one or more active dietary supplement ingredients into a mixing vessel;
introducing one or more inactive ingredients into the mixing vessel;
mixing the active ingredients and inactive ingredients to form a homogenous mixture;
after mixing the active ingredients and inactive ingredients to form a homogenous mixture, adding a water-dispersible phospholipid material to the mixture; and
blending the mixture to distribute the phospholipid component and produce liposomal particles within the composition.

2. The method of claim 1, wherein the phospholipid component is provided in an amount of no more than about 5% by weight of the composition.

3. The method of claim 1, wherein the active ingredients and inactive ingredients are mixed for about 10 to about 15 minutes to form a homogenous mixture.

4. The method of claim 1, wherein the liposomal particles have a particle size of up to about 1,000 nanometers.

5. The method of claim 1, wherein the composition comprises liposomal particles that remain stably dispersed in an aqueous solution.

6. The composition of claim 1, wherein the phospholipid is sunflower lecithin.

7. A method of preparing a dietary supplement composition, comprising:
introducing one or more dietary supplements selected from the group consisting of supplemental nutrients, metabolites, bioactive compounds, therapeutic agents, and combinations of the same as active ingredients into a mixing vessel;
adding one or more inactive ingredients selected from the group consisting of anti-caking agents, preservatives, stabilizers, and emulsifiers in effective amounts to improve the texture and shelf-life of the composition into the mixing vessel;
mixing the active ingredients and inactive ingredients to form a homogenous mixture;
after mixing the active ingredients and inactive ingredients to form a homogenous mixture, adding a water-dispersible phospholipid configured to encapsulate at least one dietary supplement to the mixture; and
blending the mixture to distribute the phospholipid and liposomally encapsulate the one or more dietary supplements within the composition
wherein the composition is operative to improve solubility and bioavailability of the dietary supplement in a human subject when consumed.

8. The method of claim 7, wherein the phospholipid particle size is up to about 1,000 nanometers as determined by dynamic light scattering analysis.

9. The method of claim 7, wherein the at least one inactive ingredients comprises a combination of one or more of silicon dioxide, polysorbate 80, propylene glycol, and acetylated monoglycerides.

10. The method of claim 7, wherein the composition comprises between about 1.5 to about 5% by weight phospholipid.

11. The method of claim 7, wherein the composition is configured for suspension in a liquid for oral consumption as a dietary supplement.

12. The method of claim 7, wherein the composition is a powdered composition configured for suspension in a liquid for oral consumption.

13. The method of claim 12, wherein the powdered composition comprises:
about 90 to about 95% by weight dietary supplement;
about 1.5 to about 3% by weight phospholipid.

14. The method of claim 13, wherein the powdered composition comprises about 1.0 to about 4.0% by weight inactive ingredients.

15. The method of claim 14, wherein the powdered composition comprises:
about 1.0 to about 2.0% by weight silicon dioxide;
about 0.1 to about 1.0% by weight polysorbate 80;
about 0.1 to about 0.5% by weight polypropylene glycol; and
about 0.1 to about 0.5% by weight acetylated monoglycerides.
